(19) United States
(12) Patent Application Publication
(10) Pub. No.: US 2002/0137043 A1
(43) Pub. Date: Sep. 26, 2002

(54) Method for reducing complexity of nucleic acid samples

(76) Inventors: Nila Patil, Woodside, CA (US); David Cox, Belmont, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 09/768,936

(22) Filed: Jan. 23, 2001

Related U.S. Application Data
(60) Provisional application No. 60/228,251, filed on Aug. 26, 2000.

(51) Int. Cl. C12Q 1/68

(52) U.S. Cl. 435/6

(57) ABSTRACT

The invention provides several methods of reducing the complexity of a population of nucleic acids prior to performing an analysis of the nucleic acids on a nucleic acid probe array. The methods result in a subset of the initial population enriched for a desired property, or lacking nucleic acids with an undesired property. The resulting nucleic acids in the subset are then applied to the array for various types of analysis. The methods are particularly useful for analyzing populations having a high degree of complexity, for example, populations of fragments spanning a human chromosome, or even a whole human genome, or mRNA populations.

### CROSS-REFERENCE TO RELATED APPLICATIONS

The present application derives priority from U.S. Ser. No. 60/228,251, filed Aug. 26, 2000, which is incorporated by reference in its entirety for all purposes.

### BACKGROUND

The scientific literature provides considerable discussion of nucleic acid probe arrays and their use in various forms of genetic analysis (for review, see Schena, Microarray Biochip Technology(Eaton Publishing, MA, USA, 2000). For example, nucleic acid probe arrays have been used for detecting variations in DNA sequences such as polymorphisms or species variations. Nucleic acid probe arrays have also been used for monitoring relative levels of populations of MRNA and detecting differentially expressed mRNAs.

Some methods for detecting polymorphisms using arrays of nucleic acid probes are described in WO 95/11995 (incorporated by reference in its entirety for all purposes). Some such arrays include four probe sets. A first probe set includes overlapping probes spanning a region of interest in a reference sequence. Each probe in the first probe set has an interrogation position that corresponds to a nucleotide in the reference sequence. That is, the interrogation position is aligned with the corresponding nucleotide in the reference sequence, when the probe and reference sequence are aligned to maximize complementarily between the two. For each probe in the first set, there are three corresponding probes from three additional probe sets. Thus, there are four probes corresponding to each nucleotide in the reference sequence. The probes from the three additional probe sets are identical to the corresponding probe from the first probe set except at the interrogation position, which occurs in the same position in each of the four corresponding probes from the four probe sets, and is occupied by a different nucleotide in the four probe sets. Such an array is hybridized to a labelled target sequence, which may be the same as the reference sequence, or a variant thereof. The identity of any nucleotide of interest in the target sequence can be determined by comparing the hybridization intensities of the four probes having interrogation positions aligned with that nucleotide. The nucleotide in the target sequence is the complement of the nucleotide occupying the interrogation position of the probe with the highest hybridization intensity.

A further strategy for detecting a polymorphism using an array of probes is described in EP 717,113. In this strategy, an array contains overlapping probes spanning a region of interest in a reference sequence. The array is hybridized to a labelled target sequence, which may be the same as the reference sequence or a variant thereof. If the target sequence is a variant of the reference sequence, probes overlapping the site of variation show reduced hybridization intensity relative to other probes in the array. In arrays in which the probes are arranged in an ordered fashion stepping through the reference sequence (e.g., each successive probe has one fewer 5′ base and one more 3′ base than its predecessor), the loss of hybridization intensity is manifested as a “footprint” of probes approximately centered about the point of variation between the target sequence and reference sequence.

Additional methods of polymorphism discovery and analysis are described in EP 0950,720. This application discusses use of primary arrays for de novo discovery of polymorphisms, and use of secondary arrays for polymorphic profiling at the newly discovered polymorphic sites of different individuals. W098/56954 discusses methods of identifying polymorphisms affecting expression of mRNA species.

Methods for using arrays of probes for monitoring expression of mRNA populations are described in U.S. Pat. No. 6,040,138,. EP 853, 679 and W097/27317. Such methods employ groups of probes complementary to mRNA target sequences of interest. An mRNA populations or an amplification product thereof is applied to such an array, and targets of interest are identified, and optionally, quantified from the extent of specific binding to complementary probes. Optionally, binding of target to probes known to be mismatched with the target can be used as a measure of background nonspecific binding and subtracted from specific binding of target to complementary probes.

U.S. Ser. No. 60/203,418, incorporated by reference for all purposes, discusses methods for determining functional regions in a genome using nucleic acid probe arrays. Additional methods for transcriptional annotation are described in, for example, U.S. Ser. No. 60/206,866 filed May 24, 2000 and 09/641,081 filed Aug. 16, 2000 incorporated by reference for all purposes.

### BRIEF DESCRIPTION OF THE FIGURES

FIG. 1 shows an exemplary scheme for removing repeat sequences from a population of nucleic acid fragments. A population of genomic DNA is digested with a restriction enzyme or DNaseI to fragments of average size 300 bp. The fragments are denatured and allowed to reanneal. Repeat sequences hybridize with each other, whereas nonrepeat sequences remain in single stranded form. The hybrids and single stranded sequences are then separated on a hydroxyapatite HPLC column. The DNA is loaded in 10 mM phosphate and eluted using a 10 mM to 1 M phosphate gradient. Single stranded DNA elutes at about 120-140 mM, and double stranded DNA elutes at about 500 mM to 1 M phosphate. The single stranded sequences are then labelled prior to application to an array.

FIG. 2 shows an exemplary scheme for enriching a tester population of nucleic acids by enrichment to a driver population of nucleic acids. In this scheme the driver DNA is a genomic clone in a BAC, YAC or PAC. The genomic DNA is cleaved to fragments of average size about 300 bp using a restriction enzyme (only one strand of double stranded fragments is shown). The fragments are ligated to linkers and amplified in the presence of a biotin labelled nucleotides. The tester DNA is a CDNA population produced by reverse transcription of an mRNA population. The EDNA is also digested with a restriction enzyme to an average length of about 300 bp. The fragments are of cDNA are ligated with linkers containing primer sites to allow amplification. The cDNA fragments are then amplified (only one strand of amplified fragments is shown). The resulting amplified cDNA fragments and biotin-labelled genomic fragments are then denatured and hybridized in solution. The genomic fragments and any hybridized cDNA are then immobilized to a streptavidin labelled magnetic bead by virtue of the affinity of the streptavidin for the biotin label on the driver nucleic acids. The hybrids are then washed to remove unhybridized tester nucleic acids. Hybridized tester nucleic acids are then dissociated from the immobilized driver by raising the temperature or lowering the salt concentration.

### DEFINITIONS

Unless otherwise apparent from the context, reference to mRNA populations includes nucleic acid populations derived therefrom by processes in which the MRNA serves as template for polynucleotide extension, such as cDNA or cRNA.

A nucleic acid is a deoxyribonucleotide or ribonucleotide polymer in either single-or double-stranded form, including known analogs of natural nucleotides unless otherwise indicated.

An oligonucleotide is a single-stranded nucleic acid ranging in length from 2 to about 500 bases.

A probe is a nucleic acid capable of binding to a target nucleic acid of complementary sequence through one or more types of chemical bonds, usually through complementary base pairing, usually through hydrogen bond formation. A nucleic acid probe may include natural (i.e. A, G, C, or T) or modified bases (e.g., 7-deazaguanosine, inosine). In addition, the bases in a nucleic acid probe may be joined by a linkage other than a phosphodiester bond, so long as it does not interfere with hybridization. Thus, nucleic acid probes may be peptide nucleic acids in which the constituent bases are joined by peptide bonds rather than phosphodiester linkages.

Specific hybridization refers to the binding, duplexing, or hybridizing of a molecule only to a particular nucleotide sequence under stringent conditions when that sequence is present in a complex mixture (e.g., total cellular) DNA or RNA. Stringent conditions are conditions under which a probe hybridizes to its target subsequence, but to no other sequences. Stringent conditions are sequence-dependent and are different in different circumstances. Longer sequences hybridize specifically at higher temperatures. Generally, stringent conditions are selected to be about 5° C. lower than the thermal melting point (Tm) for the specific sequence at a defined ionic strength and pH. The Tm is the temperature (under defined ionic strength, pH, and nucleic acid concentration) at which 50% of the probes complementary to the target sequence hybridize to the target sequence at equilibrium. (As the target sequences are generally present in excess, at Tm, 50% of the probes are occupied at equilibrium). Typically, stringent conditions include a salt concentration of at least about 0.01 to 1.0 M Na ion concentration (or other salts) at pH 7.0 to 8.3 and the temperature is at least about 30 C for short probes (e.g., 10 to 50 nucleotides). Stringent conditions can also be achieved with the addition of destabilizing agents such as formamide. For example, conditions of 5× SSPE (750 mM NaCl, 50 mM NaPhosphate, 5 mM EDTA, pH 7.4) and a temperature of 25-30 C are suitable for allele-specific probe hybridizations.

A perfectly matched probe has a sequence perfectly complementary to a particular target sequence. The test probe is typically perfectly complementary to a portion (subsequence) of the target sequence. The term “mismatch probe” refer to probes whose sequence is deliberately selected not to be perfectly complementary to a particular target sequence. Although the mismatch(s) may be located anywhere in the mismatch probe, terminal mismatches are less desirable as a terminal mismatch is less likely to prevent hybridization of the target sequence. Thus, probes are often designed to have the mismatch located at or near the center of the probe such that the mismatch is most likely to destabilize the duplex with the target sequence under the test hybridization conditions.

A polymorphic marker or site is the locus at which divergence occurs. Preferred markers have at least two alleles, each occurring at frequency of greater than 1%, and more preferably greater than 10% or 20% of a selected population. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as a the reference form and other allelic forms are designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms.

A single nucleotide polymorphism (SNP) occurs at a polymorphic site occupied by a single nucleotide, which is the site of variation between allelic sequences. The site is usually preceded by and followed by highly conserved sequences of the allele (e.g., sequences that vary in less than 1/100 or 1/1000 members of the populations).

A single nucleotide polymorphism usually arises due to substitution of one nucleotide for another at the polymorphic site. A transition is the replacement of one purine by another purine or one pyrimidine by another pyrimidine. A transversion is the replacement of a purine by a pyrimidine or vice versa. Single nucleotide polymorphisms can also arise from a deletion of a nucleotide or an insertion of a nucleotide relative to a reference allele.

### SUMMARY OF THE CLAIMED INVENTION

The invention provides methods of analyzing a subset of nucleic acids within a nucleic acid population. Such methods entail providing a population of nucleic acid fragments at least some of which have sequences that are repeated more than once in a genome. Single stranded forms of the population of nucleic acid fragments are incubated under annealing conditions, whereby single stranded forms of nucleic acid fragments having repeat sequences preferentially hybridize to each other relative to nucleic acid fragments lacking repeat sequences. Single stranded forms of the population of nucleic acid fragments are from annealed double stranded forms, the single stranded forms being enriched for nucleic acid fragments lacking repeat sequence. The separated single stranded forms of the population of nucleic acid fragments are hybridized to a nucleic acid probe array. One then determines hybridization of the probes to the single stranded forms of the population of nucleic acid fragments, thereby analyzing the fragments. In some methods, the population of nucleic acid fragments are genomic fragments, such as from the human genome. Some methods entail denaturing the population of nucleic acids fragments before the incubating step. In some methods, the separating step is performed by column chromatography. In some methods, the column is a hydroxyapatite column. In some methods, annealed and single stranded fragments elute in different fractions from the column.

In some methods, the probe array comprises a set of probes complementary to a known reference sequence, the reference sequence being the same or a variant of the sequence of a nucleic acid from which the population of nucleic acid fragments was obtained. In some methods, the determining indicates the presence of at least one variation in a fragment hybridized to the array relative to the reference sequence. In some methods, the population of nucleic acids are from a chromosome from a first individual, and the reference sequences is that of a corresponding chromosome from a second individual.

The invention also provides methods of analyzing a subset of nucleic acids within a nucleic acid population. Such methods entail providing driver and tester populations of nucleic acids. The driver and tester populations are then hybridized with each other. Nucleic acids from the tester population that hybridize to the driver population are then separated from tester nucleic acids that do not hybridize. Either the tester nucleic acids that do hybridize to the driver population, or the tester nucleic acids that do not hybridize to the driver population to a nucleic acid probe array are then further hybridized to a nucleic acid probe array. One then determines hybridization of the nucleic acid probe array to the tester nucleic acids thereby analyzing the tester nucleic acids. In some methods, the driver population of nucleic acids each bear a tag by which the driver population of nucleic acids can be immobilized to a binding moiety with affinity for the tag. For example, the tag can be biotin, and the binding moiety can be avidin or streptavidin. In some methods, the separating step is performed by immobilizing the driver population of nucleic acids and tester population of nucleic acids hybridized to the driver population via the tags of the driver population.

In some methods, the driver population of nucleic acids are a population of genomic DNA fragments, and the tester nucleic acids are a population of MRNA or nucleic acids derived therefrom. Such methods further comprise denaturing tester nucleic acids from the driver population of nucleic acids, the resulting tester nucleic acids showing reduced variance in copy number between different fragments than in the population of MRNA or nucleic acids derived therefrom. The resulting tester nucleic acids are hybridized to the array.

In some methods, the driver population of nucleic acids are genomic DNA from a first source, and the tester population of nucleic acids are genomic DNA from a second source. Such methods further comprise denaturing nucleic acids of the tester population from the driver population of nucleic acids. The resulting tester nucleic acids are enriched for tester nucleic acids having common sequences with the driver population of nucleic acids relative to the population of tester nucleic acids. The resulting tester nucleic acids are hybridized to the array.

In some methods, the tester population of nucleic acids are from a genome, and the driver population of nucleic acids are from at least one region of the genome, or a variant thereof from the same species as the genome. In some methods, at least one region is a PCR amplification product. In some methods, at least one region is cloned into a BAC, YAC or PAC. In some methods, the driver population of nucleic acids are from a plurality of noncontiguous regions of the genome or the variant thereof. In some methods, the driver population of nucleic acids are from at least ten noncontiguous regions of the genome or the variant thereof. In some methods, the method is repeated for a further population of tester nucleic acids from a further source. In some methods, the method is repeated for at least ten further populations of tester nucleic acids from at least ten further sources. Optionally, the at least ten further sources are from ten individuals in the same species. Optionally, the species is human.

In some methods, the driver population of nucleic acids are genomic DNA from a first source, and the tester population of nucleic acids are genomic DNA from a second source, and the tester nucleic acids that do not hybridize to the driver fragments are hybridized to the array, these tester nucleic acids being enriched for nucleic acids having sequences not common with sequences of the nucleic acids in the driver population.

In some methods, the driver population of nucleic acids are MRNA or nucleic acids derived therefrom, and the tester population of nucleic acids are genomic DNA. Such methods further comprise denaturing tester nucleic acids from the driver population, the resulting tester nucleic acids being enriched for genomic sequences that hybridize to the MRNA. The resulting tester nucleic acids are then hybridized to the nucleic acid probe array.

In some methods, the population of driver nucleic acids are MRNA or nucleic acids derived therefrom from a first source, and the population of tester nucleic acids are mRNA or nucleic acids derived therefrom from a second source. Such methods further comprise denaturing tester nucleic acids from the driver nucleic acids. The resulting tester nucleic acids are enriched for nucleic acids common to the two sources. The resulting tester nucleic acids are hybridized to the nucleic acid probe array. In some methods, the first and second source are from the same tissue of different species. In some methods, the first and second source are from different tissues of the same species.

In some methods, the population of driver nucleic acids are mRNA or nucleic acids derived therefrom from a first source, and the population of tester nucleic acids are mRNA or nucleic acids derived therefrom from a second sources, the tester nucleic acids that do not hybridize with the driver nucleic acids are hybridized to the array, these tester nucleic acids being enriched for sequence present in the second source and absent in the first source. In some such methods, the first and second source are from the same tissue of different species. In some such methods, the first and second source are from different tissues of the same species.

### DETAILED DESCRIPTION

I. General

The invention provides several methods of reducing the complexity of a population of nucleic acids prior to performing an analysis of the nucleic acids on a nucleic acid probe array. The methods result in a subset of the initial population enriched for a desired property, or lacking nucleic acids with an undesired property. The resulting nucleic acids in the subset are then applied to the array for various types of analysis. The methods are particularly useful for analyzing populations having a high degree of complexity, for example, populations of fragments spanning a human chromosome, or even a whole human genome, or MRNA populations.

In some methods, an initial population of nucleic acid fragments are treated so as to reduce or eliminate fragments having repeat sequences. In general, nonrepeat sequences contain the coding and key regulatory regions of genomic DNA and are of most interest for subsequent genetic analysis. Repeat sequences can be eliminated by denaturing the initial population (if double stranded), and reannealing. Single stranded forms of repeat sequences preferential hybridize with each other relative to single stranded forms of unique sequence, because there are by definition more copies of the former and therefore a greater probability of single stranded forms finding a complementary single stranded form (See, e.g., Ryffel et al., 1975, Experientia (BASEL) 31 (6) 746; Ryffel et al., 1975, Biochemistry 14(7) 1385-1389; Ryffel et al., Biochemistry 14(7) 1379-1385; Marsh et al., 1973, Biochem. Biophys. Res. Comm. 55(3) 805-811; Krueger and McCarthy, 1970, Fed. Proc. 29 (2) 757; Tereba and McCarthy, 1973, Biochem. 12(23) 4675-4679, all incorporated in their entities by reference for all purposes). After annealing, annealed and single stranded forms are separated. The resulting single stranded forms are enriched for nonrepeat sequences. These sequences are then applied to a nucleic acid probe array for a variety of genetic analyses, for example, do novo polymorphic site discovery, or detection of a plurality of predetermined polymorphic sites. In general, when analyzing the hybridization pattern of such arrays, it is desirable to discriminate between specific hybridization between complementary sequences and nonspecific hybridization between probes and sequences lacking substantial complementarity to the probes. The smaller the representation of a given target sequence in a complex mixture of sequences, the greater the ratio of nonspecific hybridization to specific hybridization between target and complementary probes. By analyzing nonrepeat sequences in the reduced presence or absence of repeat sequences, nonspecific binding of the repeat sequences to probes in the array is reduced or eliminated. Accordingly, it is possible to analyze more nonrepeat sequences simultaneously than would be the case if no steps were taken to eliminate the repeat sequences.

In other methods of the invention, a tester population of nucleic acids is enriched by virtue of its capacity to hybridize or fail to hybridize with a driver population of nucleic acids. Typically, the driver population of nucleic acids bears a tag that can be immobilized via a binding moiety having specific affinity for the tag. The driver population can be immobilized before, after or during hybridization to the tester population. Nucleic acids from the tester population that hybridize to the driver population are thereby also immobilized via their association with the immobilized driver nucleic acids. After hybridization and immobilization of driver nucleic acids and associated tester nucleic acids, the solution phase containing unhybridized tester nucleic acids is separated from the immobilized phase. In some methods, tester nucleic acids from the solution phase are then applied to an array. These tester nucleic acids are deficient at least relative to the initial population of tester nucleic acids for nucleic acids that hybridize with the driver population.

In other methods, the solution phase is removed, and tester nucleic acids associated with driver nucleic acids are dissociated. The driver nucleic acids remain immobilized but the resulting tester nucleic acids are in solution. These tester nucleic acids are then applied to an array. These nucleic acids are enriched for tester nucleic acids that hybridize to the driver nucleic acids. As discussed in more detail below, there are a variety of permutations of driver and tester populations that can be used in these methods. The nature of the subsequent genetic analysis after application of the array of course depends on the nature of the driver and tester populations, and which tester fragments (i.e., whether hybridizing or not hybridizing to the driver nucleic acids) are retained.

II. Method of Removing Repeat Sequences

Repeat sequences are sequences occurring occur more than once in a haploid genome of a single organism. In some instances, multiple copies of a repeat sequence are identical. In other instances, there are some divergences between copies but substantial sequence identity, e.g., (at least 80 or 90%). More than 30% of human DNA consists of sequences repeated at least 20 times. Families of repeated DNA sequences of 100-500 bp that are interspersed throughout the genome are sometimes known as SINES (short interspersed repeats). Alu sequences are examples of SINES that are about 300 bp and occur almost 1 million times in the human genomes. Longer interspersed repeat sequences of 1 kb or more are known as LINES (long interspersed repeats). Some repeat sequences are not interspersed throughout the genome but are concentrated at particular loci. These repeats are known as satellite repeats. Some repeats sequences include genes such as genes for ribosomal RNAs and histones. However, the function, if any, of most repeat sequences is unclear. The vast majority of protein coding sequences and their associated regulatory sequences occur in single copy regions of the genome.

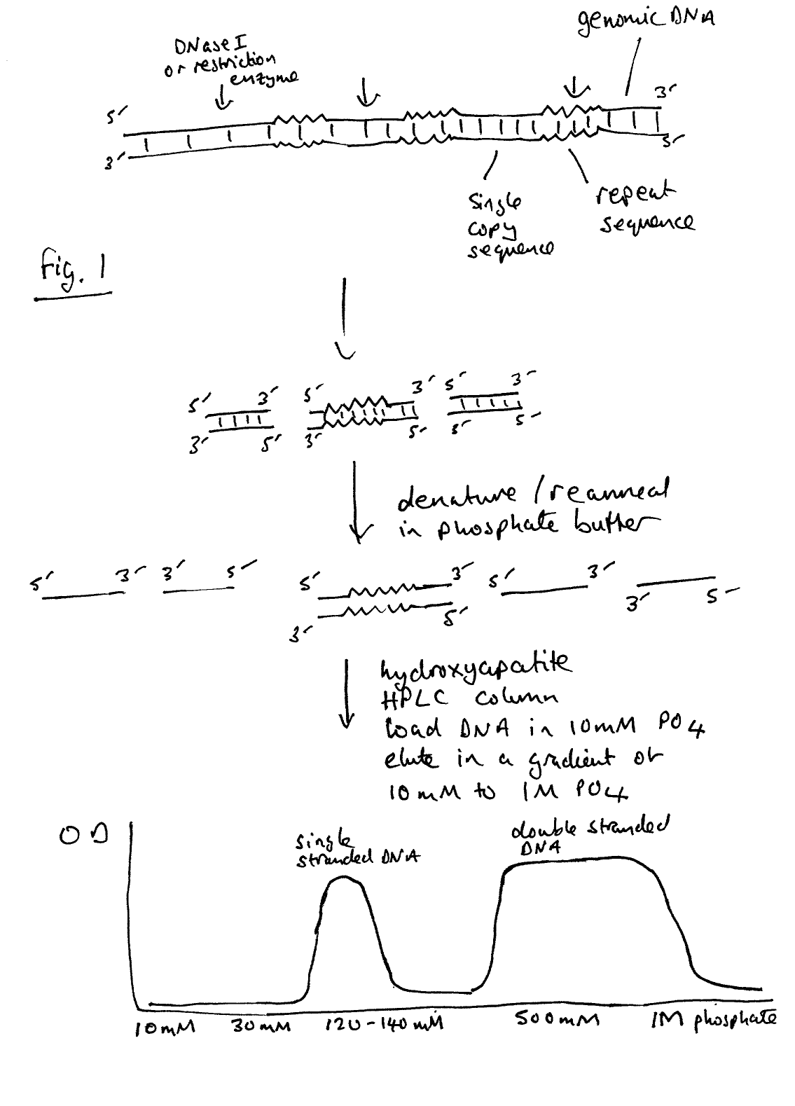

The present invention provided methods for enriching for single copy regions of a genome relative to repeat sequences before performing a genetic analysis using a nucleic acid probe array (see FIG. 1). The starting population of fragments for enrichment can be from a whole genome, a collection of chromosomes therefrom, a single chromosome, or one or more regions from one or more chromosomes. In some methods, the fragments are overlapping fragments spanning a length of 100 kb, 1 Mb, 10 Mb or 100 Mb. Typically, the fragments are from obtained from the same individual. The individual can be a human or other mammal or other eukaryotic species. The methods are not generally necessary for analysis of prokaryotic DNA due to lack of substantial numbers of repeat sequences in prokaryotic DNA. The fragments can be obtained from any tissue sample containing genomic DNA from an individual.

The fragments are produced by fragmenting an initial substrate such as an isolated chromosome or genome. Optionally, the initial substrate can be amplified, and/or labelled before fragmentation. Both enzymatic and mechanic methods can be used for fragmentation. The fragmenting can be effected by restriction digestion, often using a partial digest with a four-bp cutting enzyme, or a digest with a mixture of enzymes or with DNaseI. Alternatively, fragments can be produced by sonication, or by PCR amplification using random primers or random fragments of an initial substrate. Other suitable methods include mechanic or liquid shearing by using a French press or a UCHGR Shearing Device. In some methods, fragments are attached to linkers at one or both ends to provide primer sites for subsequent amplification. In some methods, fragments have an average size of about 300 bp. For example, appropriate restriction enzymes may be used to cut genomic DNAs to a desired range of sizes. Fragments containing repeat sequences are removed from the population by a combination of denaturation (assuming the fragments are double stranded) and reannealing. Denaturation can be effected by heating fragments in excess of the averaging melting point. Fragments are then cooled to below the melting point (e.g., about 25 degrees below the melting point) for reannealing. The reassociation can be followed by monitoring hyperchromicity at 260 nm. As DNA renatures, the hyperchromicity increases due to greater absorbance of double stranded relative to single stranded DNA. The hyperchromicity curve shows a point of inflexion at which half of the DNA is reannealed. The reannealing reaction is often stopped about this time, but the duration of the reaction can be adjusted depending on the percentage of repeat DNA in the sample. The more repeat DNA the longer the annealing reaction should proceed. The reannealing reaction can effectively be stopped by rapid cooling of the annealing mixture to just above freezing.

After the annealing reaction, annealed double stranded DNA is separated from single stranded DNA. Separation can be effected using column chromatography. A hydroxyapatite (calcium phosphate) column is particularly suitable (see Ryffel & McCarthy, Biochemistry, 14.7, 1385-1389 (1975) incorporated by reference for all purposes. Both single and double stranded forms bind to the column at low phosphate concentration (10-30 mM sodium phosphate). At intermediate concentrations (120 mM to 140 mM, single stranded DNA passes through and double stranded DNA binds. At higher concentrations (400 mM), both single and double stranded DNA pass through. DNA can be loaded on the column at low phosphate concentration, in which case both single and double stranded forms bind. Single stranded forms are then eluted with an increasing gradient of sodium phosphate concentration. Alternatively, single and double stranded forms can be loaded at intermediate phosphate concentration, in which case the single stranded form passes though without binding and the double stranded form binds (see Genome Analysis: A Laboratory Manual, Volume 2, Detecting Genes (Eds. Bruce Birren et al., Cold Spring Harbor Press, 1998). In some methods, hydroxyapatite columns are combined with HPLC. Alternatively, or additionally, the annealing reaction mixture can be treated with a nuclease that selectively digests double stranded DNA relative to single stranded.

After separation of single stranded forms, the single stranded forms can be applied directly to an array, or can be the subject of additional treatment before applying to an array. For example, in some methods, the single stranded fragments are allowed to anneal with each other, forming double stranded fragments, which are then amplified and labelled, and denatured before being applied to the array. In some methods, single stranded forms that have not previously been labelled are now labelled before applying to an array. Some methods for end-labelling fragments are described by W097/27317. In some methods, the single stranded fragments, optionally after renaturation to double stranded fragments, are broken down to still smaller fragments, before being applied to an array.

The type of array to which the fragments are applied of course depends on the form of contemplated analysis. In some methods, fragments are applied to arrays designed for de novo polymorphisms discovery. These arrays typically contain overlapping probes tiling a region of a known reference sequence. The hybridization pattern of the fragments to the array indicates the site and nature of points of divergence between the sequence of the fragments and the reference sequence, and hence the location and identity of polymorphic sites. In other methods, the fragments are applied to an array designed to detected a collection of polymorphisms whose location and nature of polymorphic forms is already known. In such methods, the hybridization pattern of the nucleic acid fragments to the array indicates a polymorphic profile of the individual from whom the fragments were obtained (i.e., a matrix of polymorphic sites, and polymorphic forms present in those sites).

III. Other Methods of Reducing Sample Complexity

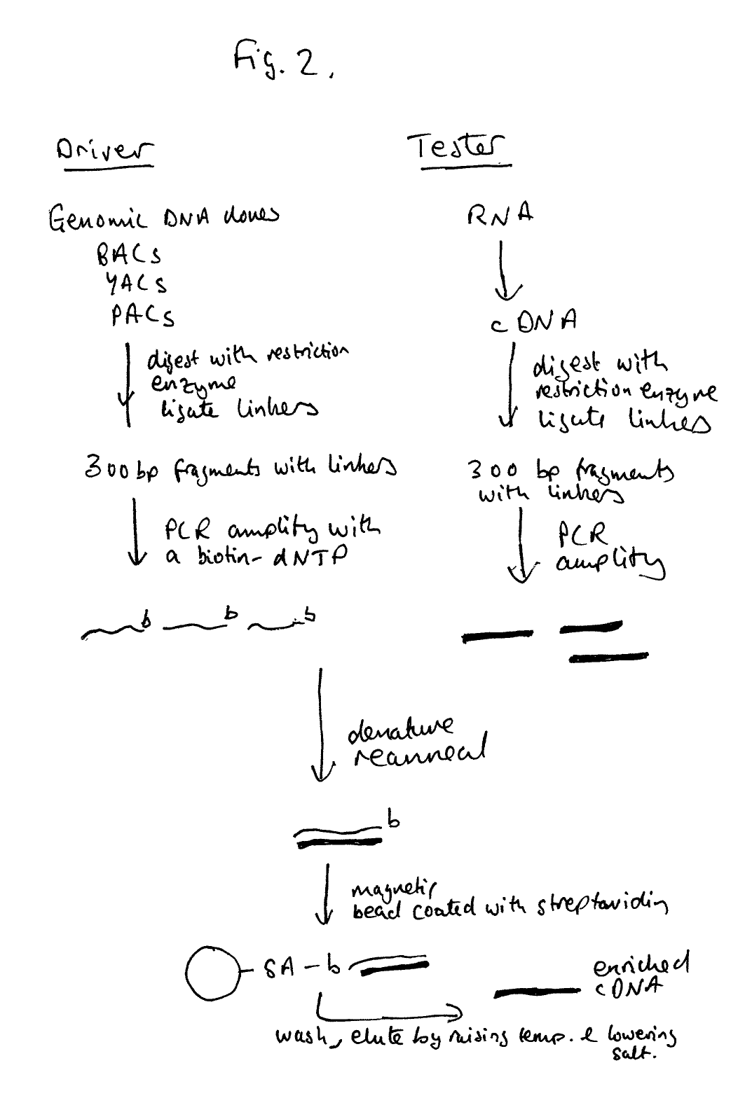

A variety of enrichments can be performed by hybridization of tester nucleic acids to driver nucleic acid as described above (see FIG. 2). In these methods, either or both of driver and tester nucleic acids can be amplified before performing the enrichment procedure. Optionally, driver and/or tester are fragmented before performing hybridization. Fragments can be achieved by any of the methods described above, usually to an average size of about 300 bp. Fragmentation before enrichment is typical with genomic populations and possible, but not usual, with mRNA populations. In some methods, a population of nucleic acids is fragmented, the fragments are ligated to oligonucleotides to provide primer sites, and the resulting fragments are amplified. The tester nucleic acid fragments can also be labelled. Labelling can be performed before or after the enrichment procedure. In these methods, populations of driver and tester nucleic acid fragments are denatured (if initially double stranded), mixed (if denaturation was performed separately for each population) and allowed to reanneal. As in the methods for eliminating repeat sequences, denaturation can be performed by raising the temperature over the average melting point of driver and tester nucleic acid populations. The two populations can be denatured separately or together. Hybrids between tester and driver nucleic acids are separated from unhybridized tester nucleic acid. Separation can be effected by inclusion of a tag on all driver fragments and immobilizing the driver fragments to a binding moiety. For example, a biotin tag can be attached to driver fragments by amplifying them using a biotin labelled primer or biotin labelled nucleotides or by ligating them to a biotin labelled oligonucleotide or by directly attaching biotin to the fragments (see e.g., Birren et al. supra, at ch. 3). Biotin labelled driver fragments can then be immobilized to a support bearing an avidin or streptavidin binding moiety For example, magnetic beads coated with streptavidin, available from Dynal, Norway, are suitable for immobilizing biotin-labelled DNA. Procedures for performing enrichments of cDNA using immobilized DNA on beads are described by Birren et al., supra at Ch. 3. Other combinations of tag and binding moiety can similarly be used. Alternatively, hybrids can be separated from single stranded fragments using hydroxyapatite chromatography as described above. Alternatively, separation can be effected using a nuclease that digests duplex nucleic acids without digesting single stranded nucleic acids or vice versa. For example, S1 nuclease preferentially digests single stranded DNA, whereas most restriction enzymes preferentially digest double stranded DNA.

1. Driver Population is Genomic and Tester Population is mRNA

In some methods, the driver population is genomic DNA and the tester population is an mRNA population or nucleic acid population derived therefrom (e.g., cDNA or cRNA). As will become apparent, such methods serve to normalize the representation of different species within the mRNA population (or nucleic acids derived therefrom). In other words, the methods enrich the representation of rare mRNA species relative to the more common mRNA species. In such methods, the driver population can be from a whole genome, a chromosome, a collection of chromosomes or one or more regions of one or more chromosomes. If an entire genome is included, then the enriched population of mRNAs includes mRNAs spread throughout the genome. If a single chromosome is included, then the enriched population of mRNAs is restricted to mRNAs hybridizing to that chromosome, and so forth. The mRNA population used as the tester population can be from a single tissue type, from a cell line or from a mixture of tissue types. If from a single tissue type, the mRNA population and the resulting enriched population contains a bias toward the mRNAs expressed in that cell type. If the mRNA population is from a representative mixture of tissue types, then the population and the subsequent enriched populations contains most or substantially all (e.g., at least 50%, 75% or 90%) of mRNAs expressed by the organism. Some cell lines, such as HeLa cells, also express a substantial proportion of all mRNAs typically expressed in an organism. If cDNA or cRNA is prepared from mRNA, the preparation can be performed under conditions that preserve the relative representations of mRNA species in the original population as described by 6,040,138. However, such is generally not necessary because the proportions are, of course, deliberately changed in the enrichment procedure. Thus, conventional methods of cDNA preparation using polyT primers or random hexamers can be used (See Birren et al., supra at ch. 3). In some methods, adapters are ligated to cDNA to facilitate subsequent amplification or labelling.

When driver genomic DNA is hybridized with tester MRNA (or a nucleic acid derived therefrom), the mRNA hybridizes to complementary sequences in the genomic DNA sequences. However, in general, each MRNA species has only a single complementary genomic DNA sequence in a haploid genome. Accordingly, highly represented MRNA species and minimally represented species (and intermediately represented sequences) in generally all hybridize to genomic DNA to a similar extent. In theory, one molecule of MRNA should hybridize per haploid genome for a single copy gene. In practice, this ratio is not observed for all single copy genes due to the presence of introns. For example, a gene having ten spaced exons can hybridize to different regions of ten copies of the same mRNA. Nevertheless, the hybridization does represent in substantial normalization between mRNA species. For example, whereas the variation copy number between species in an unnormalized population can be greater than 10⁵ in a normalized population, the variation is more typically within a factor of 10, 100, or 1000.

After performing hybridization, hybrids between tester and driver populations are separated from unhybridized tester. The unhybridized tester is set aside. Tester nucleic acids are then dissociated from driver nucleic acids (e.g., by raising the temperature above the melting point). The drive nucleic acids remain associated with the solid phase, and the resulting tester nucleic acids are obtained in solution. The resulting tester nucleic acids are initially in single stranded form. Optionally, the single stranded fragments can be labelled (if not labelled already) and applied directly to an array. Alternatively, the fragments can be renatured with each other, for amplification, and optionally labelling. Amplified fragments are then denatured again before being applied to an array.

The resulting testing fragments can be subject to a variety of genetic analyses. In some methods, the fragments are used for de novo polymorphism discovery, in similar fashion to that described above. The polymorphisms thereby discovered necessarily occur within expressed regions of the genome. The resulting tester fragments can also be used for polymorphic profiling of previously characterized polymorphic sites within expressed regions within an individual. Use of mRNA populations has advantages relative to use of genomic DNA in that nonexpressed regions of the genome, which probably contain relatively few polymorphic sites of functional significance, but which would otherwise contribute to a background of nonspecific binding on the array are not applied to the array. It is estimated that only 5% of the human genome contains coding regions.

The resulting tester fragments can also be used for discovering relatively rare differentially expressed genes. For example, by comparing tester populations, enriched as described above from different tissue types, one can identify species within one tester population that are not expressed within another. Such mRNA species can be cloned as described in W097/27317. This type of analysis is particularly useful for identifying genes that are expressed at a low level in one tissue, and not at all in another tissue.

2. Driver and Tester Populations are both Genomic

In some methods, both driver and tester populations are genomic but from different sources. In some methods, the different sources are different individuals from the same species, or individuals from different species. For example, the two sources can be two different humans, or one human and one cat, or one mouse and one dog, and so forth. Such methods serve to enrich either fragments that are common to the two sources or fragments that differ between the two sources. For the former type of enrichment, one retains tester fragments hybridizing to driver fragments. For the latter type of enrichment, one retains tester fragments not hybridizing to driver fragments. Common sequences are of interest because commonality often implies evolutionary conservation and therefore an important functional role. Polymorphisms occurring within regions that are conserved between species are more likely to have phenotypic consequences. Accordingly, given the vast number of polymorphic sites within a genome, it can be advantageous to focus on conserved regions for polymorphism discovery, and/or to use polymorphisms within conserved regions for association studies. Disparate sequences between sources are also of interest, because these sequences are the locus of genetic diversity between different individuals and/or species.

In these, as in other methods, driver and tester populations can be obtained from whole genomes, collections of chromosomes, individual chromosomes or one or more regions of individual chromosomes. Usually, the fragments within a driver population are obtained from the same individual, as is the case for fragments within a tester population. However, the driver and tester populations are generally obtained from different individuals. Either driver and/or tester populations can be amplified before performing hybridization. The tester population can be labelled before or after the hybridization. If the goal is to isolate common fragments between the driver and tester population, nonhybridizing fragments from the tester population are set aside, and tester fragments hybridizing to the driver are dissociated from the driver. Optionally, these fragments can be subject to amplification and/or labelling before being applied to an array. If the goal is to isolate disparate fragments between the driver and tester population, then hybridizing driver and tester fragments are set aside. The nonhybridizing tester fragments can be directly applied to an array (optionally with labelling, if not already labelled). Alternatively, the nonhybridizing tester fragments can be hybridized with each other, amplified, and optionally, labelled before being applied to an array.

In other methods, hybridization between driver and tester fragments is used as a surrogate for selective amplification of a certain region of genomic DNA. The goal in such methods is to apply one or more regions of genomic DNA to an array without applying others. Such could be achieved by selective amplification of the desired regions. However, performing selective amplification on a large number of samples, particularly if the amplification is a multiplex amplification of multiple noncontiguous regions can be tedious and subject to error. Alternatively, the amplification can be performed on a single genomic sample, and the amplified sample then used as a driver population to enrich equivalent regions from a broader initial population of tester DNA. For example, the driver population can be a long range PCR product of a particular chromosome, or a YAC or BAC clone within a particular chromosome. The tester population can be a whole genomic population or the whole chromosome from which the BAC, YAC or long range PCR product was obtained. When the tester population is annealed with the driver population, substantially only the complementary fragments within the tester population hybridize. These fragments can then be dissociated from the driver and applied to an array (optionally with labelling, if not already labelled). The fragments can be used for de novo polymorphism discovery or polymorphic profiling as described in other methods. The benefits of such enrichment are particularly evident when it desired to analyze a plurality of noncontiguous regions within a genome (e.g., ten or more), and/or when it desired to analyze tester DNA from a plurality of individuals (e.g., ten or more).

c. Driver Population mRNA Tester Population Genomic DNA

In other methods, a driver population of mRNA or nucleic acids derived therefrom is used to enrich a tester population of genomic DNA. Such methods enrich the genomic DNA population for fragments represented in the mRNA. The enrichment results in a population of nucleic acids that are normalized in copy number relative to the original population of MRNA. In addition, the enriched nucleic acids include regions of genomic DNA proximate to expressed regions, such as intron-exon borders, and nonexpressed regulatory sequences, such as promoters and enhancers. The enriched population can be used in similar analyses to those described in section III(a) above. In addition, the population is useful for discovering and detecting polymorphisms in nonexpressed regions of DNA that cannot be detected by analysis of MRNA populations. Such polymorphism can have roles in regulating the extent of expression of a gene.

The tester population can be from a whole genome, a chromosome, a collection of chromosomes or one or more regions of one or more chromosomes. If an entire genome is included, then the enriched population of nucleic acids typically includes nucleic acids spread throughout the genome. If a single chromosome is included, then the enriched population of nucleic acids is of course within this chromosome. The mRNA population used as the driver population can be from a single tissue type, from a cell line or from a mixture of tissue types, as described in section III(a). After hybridization of driver and tester populations, unhybridized tester fragments are set aside. Hybridized tester fragments are dissociated from the driver fragments. The resulting tester fragments can the be applied to an array (optionally with labelling, if not already labelled). Alternatively, the resulting tester fragments can be renatured, amplified, and optionally, labelled before being applied to an array.

d. Driver and Tester Populations are mRNA

In some methods, both driver and tester populations are mRNA populations from different sources. The different sources can be different tissues from an individual or individuals within the same species. Alternatively, the different sources can be the same tissue type from different species, (e.g., human and mouse, cat, dog, horse, cow, sheep, primate and so forth). In a further variation, the two sources can be the same tissue subject to different environmental factors, for example, exposure to a drug or potentially toxic compound. The enrichment can be used to enrich either for fragments that are common to the two populations or for fragments that are differentially represented between the two populations. Fragments that are common to the two populations of MRNA from the different sources are enriched for sequences that have been subject to evolutionary conservation. As previously discussed, polymorphisms within such sequences are particularly likely to have phenotypic consequences on an organisms. Accordingly, such common species are useful for de novo polymorphism discovery and profiling of previously characterized polymorphisms. Differentially expressed mRNA species can also be used for polymorphism analysis. Alternatively, such mRNA species can be applied to expression monitoring arrays for identification and further characterization of the genes encoding such species. For example, such mRNA species can be applied to probe arrays containing large numbers of random probes. Probes showing specific hybridization can then be used as primers or probes to isolate genes responsible for differentially expressed mRNAs. Alternatively, the mRNA species can be hybridized to an expression monitoring array containing probes for known mRNA species. If the mixture of differentially expressed mRNAs resulting from enrichment is one of the known mRNA species, this is indicated by the resulting hybridization pattern.

As in other methods, common mRNA species between the two populations are isolated by setting aside nonhybridizing tester mRNA and dissociating hybridizing tester mRNA from driver mRNA. Optionally, the dissociated tester mRNA can be subjected to amplification and labelling before applying to an array . Amplification, if any, can be conducted with or without preservation of relative copy number of amplified species.

IV. Modes of Practicing the Invention

a. Probe Arrays

As previously discussed a variety of probe array designs can be used in the invention depending on the intended type of genetic analysis. Probe arrays and their uses are reviewed in Schena, Microarray Biochip Technology(Eaton Publishing, MA, USA, 2000). Some arrays are designed for de novo discovery of polymorphisms. Such arrays contain at least a first set of probes that tiles one or more reference sequences (or regions of interest therein). The reference sequence can be a chromosome, a genome, or any part thereof. Tiling means that the probe set contains overlapping probes, which are complementary to and span a region of interest in the reference sequence. For example, a probe set might contain a ladder of probes, each of which differs from its predecessor in the omission of a 5′ base and the acquisition of an additional 3′ base. The probes in a probe set may or may not be the same length. Such arrays typically contain at least one probe for each base to be analyzed.

Such an array is hybridized to target nucleic acid samples prepared by one of the enrichment methods described above and/or to a control sample known to contain the reference sequence(s) tiled by the array. Optionally, the array can by hybridized simultaneously to more than one target sample or to a target sample and reference sequence by use of two-color labelling (e.g., the reference sequence bears one label and a target sample bears a second label). If the array is hybridized to a control reference sequence (or a target sequence that is identical to the reference sequence), all probes in the first probe set specifically hybridize to the reference sequence. If the array is hybridized to a target sample containing a target sequence that differs from the reference sequence at a polymorphic site, then probes flanking the polymorphic site do not show specific hybridization, whereas other probes in the first probe set distal to the polymorphic site do show specific hybridization. The existence of a polymorphism is also manifested by differences in normalized hybridization intensities of probes flanking the polymorphism when the probes hybridized to corresponding targets from different individuals. For example, relative loss of hybridization intensity in a “footprint” of probes flanking a polymorphism signals a difference between the target and reference (i.e., a polymorphism) (see EP 717,113, incorporated by reference in its entirety for all purposes). Additionally, hybridization intensities for corresponding targets from different individuals can be classified into groups or clusters suggested by the data, not defined a priori, such that isolates in a give cluster tend to be similar and isolates in different clusters tend to be dissimilar. See WO 97/29212 (incorporated by reference in its entirety for all purposes).

Optionally, primary arrays of probes can also contain second, third and fourth probe sets as described in WO 95/11995. The probes from the three additional probe sets are identical to a corresponding probe from the first probe set except at the interrogation position, which occurs in the same position in each of the four corresponding probes from the four probe sets, and is occupied by a different nucleotide in the four probe sets. After hybridization of such an array to a labelled target sequence, analysis of the pattern of label revealed the nature and position of differences between the target and reference sequence. For example, comparison of the intensities of four corresponding probes reveals the identity of a corresponding nucleotide in the target sequences aligned with the interrogation position of the probes. The corresponding nucleotide is the complement of the nucleotide occupying the interrogation position of the probe showing the highest intensity.

Optionally, arrays for de novo polymorphism detection can tile both strands of reference sequences. Both strands are tiled separately using the same principles described above, and the hybridization patterns of the two tilings are analyzed separately. Typically, the hybridization patterns of the two strands indicates the same results (i.e., location and/or nature of polymorphic form) increasing confidence in the analysis. Occasionally, there may be an apparent inconsistency between the hybridization patterns of the two strands due to, for example, base-composition effects on hybridization intensities. Such inconsistency signals the desirability of rechecking a target sample either by the same means or by some other sequencing methods, such as use of an ABI sequencer.

Arrays used for analyzing previously identified polymorphisms typically differ from the arrays for de novo identification in the following respects. First, whereas probes are typically included to span the entire length of a reference sequence in de novo discovery arrays, in arrays for analyzing precharacterized polymorphisms only a segment of a reference sequence containing a polymorphic site and immediately flanking bases is typically spanned in secondary arrays. For example, this segment is often of a length commensurate with that of the probes. Second, an array for analyzing precharacterized polymorphisms typically includes at least two groups of probes. A first group of probes is designed based on the reference sequence, and the second group based on a polymorphic form thereof. If there are three polymorphic forms at a given polymorphic site, a third group of probes can be included. Finally, because fewer probes are generally required to analyze precharacterized polymorphisms than in the de novo identification of polymorphisms, the former arrays often are designed to detect more different polymorphic sites than primary arrays. For example, whereas a de novo polymorphism discovery array may tile a single chromosome, an array for analyzing precharacterized polymorphisms can easily analyze 1,000, 10,000, 100,000 or 1,000,000 polymorphic sites in reference sequences dispersed throughout the human genome.

The design of suitable probe arrays for analysis of predetermined polymorphisms and interpretation of the hybridization patterns is described in detail in WO 95/11995; EP 717,113; and WO 97/29212. Such arrays typically contain first and second groups of probes, which are designed to be complementary to different allelic forms of the polymorphism. Each group contains a first set of probes, which is subdivided into subsets, one subset for each polymorphism. Each subset contains probes that span a polymorphism and proximate bases and are complementary to one allelic form of the polymorphism. Thus, within the first and second probe groups there are corresponding subsets of probes for each polymorphism. The hybridization patterns of these probes to target samples can be analyzed by footprinting or cluster analysis, as described above. For example, if the first and second probes groups contain subsets of probes respectively complementarity to first and second allelic forms of a polymorphic site spanned by the probes, then on hybridization of the array to a sample that is homozygous for the first allelic form all probes in the subset from the first group show specific hybridization, whereas probes in the subset from the second group that span the polymorphism show only mismatch hybridization. The mismatch hybridization is manifested as a footprint of probe intensities in a plot of normalized probe intensity (i.e., target/reference intensity ratio) for the subset of probes in the second group. Conversely, if the target sample is homozygous for the second allelic form, a footprint is observed in the normalized hybridization intensities of probes in the subset from the first probe group. If the target sample is heterozygous for both allelic forms then a footprint is seen in normalized probe intensities from subsets in both probe groups although the depression of intensity ratio within the footprint is less marked than in footprints observed with homozygous alleles.

Alternatively, the first and second groups of probes can contain first, second, third and fourth probe sets. Each of the probe sets can be subdivided into subsets, one for each polymorphism to be analyzed by the array. The first set of probes in each group is spans a polymorphic site and proximate bases and is complementary to one allelic form of the site. The second, third and fourth sets, each have a corresponding probe for each probe in the first probe set, which is identical to a corresponding probe from the first probe set except at the interrogation position, which occurs in the same position in each of the four corresponding probes from the four probe sets, and is occupied by a different nucleotide in the four probe sets.

Arrays for analyzing precharacterized polymorphisms are interpreted in similar manner to the arrays for polymorphism discovery having four sets of probes described above. For example, consider an array having first and second groups of probes, each having four sets of probes designed based on first and second allelic forms of a single polymorphic site hybridized to a target containing homozygous first allele. The probes from the first probe set of the first group all show perfect hybridization to the target sample, and probes from other probe sets in the first group all show mismatch hybridization. All probes from the second group of probes show at least one mismatch except one of the four corresponding probes having an interrogation position aligned with the polymorphic site. A probe from the second, third or fourth probes sets probes having an interrogation position occupied by a base that is the complement of the corresponding base in the first allelic form shows specific hybridization.

If such an array is hybridized to a target sample containing homozygous second allelic form, the mirror image hybridization pattern is observed. That is all probes in the first probe set of the second group show matched hybridization, and probes from the second, third and fourth probe sets in the second probe group show mismatch hybridization. All but one probe in the first group of probes shows mismatch hybridization. The one probe showing perfect hybridization has an interrogation site aligned with the polymorphic site and occupied by the complement of the base occupying the polymorphic site in the second allelic form.

If such an array is hybridized to a target sample containing heterozygous first and second allelic forms, the aggregate of the above two hybridization patterns is observed. That is, all probes in the first probe set from both the first and second group show perfect hybridization (albeit with reduced intensity relative to a homozygous target), and one additional probe from the second, third or fourth probe set in each group shows perfect hybridization. In each group, this probe has an interrogation position aligned with the polymorphic site and occupied by a base occupying the polymorphic site in one or other of the allelic forms.

Typically, arrays for analyzing precharacterized polymorphisms contain multiple subsets of each of the probe sets described, with a separate subset for each polymorphism. Thus, for example, a secondary array for analyzing a thousand polymorphisms might contain first and second groups of probes, each containing four probe sets, with each of the four probe sets, being divided into 1000 subsets corresponding to the 1000 different polymorphisms. In this situation, analysis of the hybridization patterns from four subsets relating to any given polymorphisms is independent of any other polymorphism. Analysis of the hybridization pattern of such an array to a target sample indicates which polymorphic form is present at some or all of the polymorphic sites represented on an array. Thus, the individual is characterized with a polymorphic profile representing allelic variants present at a substantial collection of polymorphic sites.

Methods for using arrays of probes for monitoring expression of mRNA populations are described in PCT/US96/143839, WO 97/17317, and U.S. Pat. No. 5,800,992. Some methods employ arrays having nucleic acid probes designed to be complementary to known mRNA sequences. An mRNA populations or nucleic acids derived therefrom are applied to such an array, and targets of interest are identified, and optionally, quantified from the extent of specific binding to complementary probes. Optionally, binding of target to probes known to be mismatched with the target can be used as a measure of background nonspecific binding and subtracted from specific binding of target to complementary probes. Some methods employ arrays of random or arbitrary probes (also known as generic arrays). Such probes hybridize to complementary MRNA sequences present in a population, and are particularly useful for identifying and characterizing hitherto unknown mRNA species.

2. Synthesis and Scanning of Probe Arrays

Arrays of probe immobilized on supports can be synthesized by various methods. Methods of forming arrays of nucleic acids, peptides and other polymer sequences are disclosed in, for example, U.S. Pat. Nos. 5,143,854, 5,252,743, 5,384,261, 5,405,783, 5,424,186, 5,429,807, 5,445,943, 5,510,270, 5,677,195, 5,571,639, 6,040,138, all incorporated herein by reference for all purposes. The oligonucleotide analogue array can be synthesized on a solid substrate by a variety of methods, including light-directed chemical coupling, and mechanically directed coupling. See U.S. Pat. No. 5,143,854, WO 90/15070) and Fodor et al., WO 92/10092 and WO 93/09668 and U.S. Pat. Nos. 5,677,195, 6,040,193, and 5,831,070, U.S. Ser. No. 60/203,418, McGall et al., U.S. Ser. No. 08/445,332; U.S. Pat. No. 5,143,854; EP 476,014). Such arrays typically have at least 1000, 10,000, 100,000 or 1,000,000 different probes occupying 1000 different regions within a square centimeter. Algorithms for design of masks to reduce the number of synthesis cycles are described by Hubbel et al., U.S. Pat. No. 5,571,639 and U.S. Pat. No. 5,593,839. Arrays can also be synthesized in a combinatorial fashion by delivering monomers to cells of a support by mechanically constrained flowpaths. See Winkler et al., EP 624,059. Arrays can also be synthesized by spotting monomers reagents on to a support using an ink jet printer. See id.; Pease et al., EP 728,520. Arrays can also be synthesized by spotting preformed nucleic acid probes on to a substrate, as described by Winkler et al., EP 624,059. Such nucleic acid can be covalently attached or attached via noncovalent linkage, such as biotin-avidin or biotin-streptavidin. Alternatively, the DNA can be held in place by coating the surface of an array with polylysine, which is positively charged and binds to negatively charged DNA. Nucleic acid probe arrays of standard or customized types are also commercially available from Affymetrix.

After hybridization of control and target samples to an array containing one or more probe sets as described above and optional washing to remove unbound and nonspecifically bound probe, the hybridization intensity for the respective samples is determined for each probe in the array. For fluorescent labels, hybridization intensity can be determined by, for example, a scanning confocal microscope in photon counting mode. Appropriate scanning devices are described by e.g., Trulson et al., U.S. Pat. No. 5,578,832; Stern et al., U.S. Pat. No. 5,631,734. Such devices are commercially available from Affymetrix.

3. Reference Sequences

Reference sequences for polymorphic site identification are often obtained from computer databases such as Genbank, the Stanford Genome Center, The Institute for Genome Research and the Whitehead Institute. The latter databases are available at http://www-genome.wi.mit.edu; http://shgc.stanford.edu and http://ww.tigr.org. A reference sequence can vary in length from 5 bases to 100,000, 1 Mb, 10 Mb, 100 Mb or 1 GB bases. Reference sequences can be genomic DNA or episomes. In some methods, reference sequences are mRNA.

4. Nucleic Acid Sample Preparation

The nucleic acid samples hybridized to arrays can be genomic, RNA or cDNA. Optionally, nucleic acid samples can be subject to amplification before or after enrichment. An individual genomic DNA segment from the same genomic location as a designated reference sequence can be amplified by using primers flanking the reference sequence. Multiple genomic segments corresponding to multiple reference sequences can be prepared by multiplex amplification including primer pairs flanking each reference sequence in the amplification mix. Alternatively, the entire genome can be amplified using random primers (typically hexamers) (see Barrett et al., Nucleic Acids Research 23, 3488-3492 (1995)) or by fragmentation and reassembly (see, e.g., Stemmer et al., Gene 164, 49-53 (1995)). Genomic DNA can be obtained from virtually any tissue source (other than pure red blood cells). For example, convenient tissue samples include whole blood, semen, saliva, tears, urine, fecal material, sweat, buccal, skin and hair. RNA samples are also often subject to amplification. In this case amplification is typically preceded by reverse transcription. Amplification of all expressed mRNA can be performed, for example, as described by commonly owned WO 96/14839 and WO 97/01603.

5. Methods of Amplification

The PCR method of amplification is described in PCR Technology: Principles and Applications for DNA Amplification (ed. H.A. Erlich, Freeman Press, NY, N.Y., 1992); PCR Protocols: A Guide to Methods and Applications (eds. Innis, et al., Academic Press, San Diego, Calif., 1990); Mattila et al., Nucleic Acids Res. 19, 4967 (1991); Eckert et al., PCR Methods and Applications 1, 17 (1991); PCR (eds. McPherson et al., IRL Press, Oxford); and U.S. Pat. No. 4,683,202 (each of which is incorporated by reference for all purposes). Nucleic acids in a target sample can be labelled in the course of amplification by inclusion of one or more labelled nucleotides in the amplification mix. Labels can also be attached to amplification products after amplification e.g., by end-labelling. The amplification product can be RNA or DNA depending on the enzyme and substrates used in the amplification reaction.

Other suitable amplification methods include the ligase chain reaction (LCR) (see Wu and Wallace, Genomics 4, 560 (1989), Landegren et al., Science 241, 1077 (1988), transcription amplification (Kwoh et al., Proc. Natl. Acad. Sci. USA 86, 1173 (1989)), and self-sustained sequence replication (Guatelli et al., Proc. Nat. Acad. Sci. USA, 87, 1874 (1990)) and nucleic acid based sequence amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively.

V. Uses of Genetic Analyses

1. Polymorphic Profiling

a. Association Studies and Diagnosis

The polymorphic profile of an individual may contribute to phenotype of the individual in different ways. Some polymorphisms occur within a protein coding sequence and contribute to phenotype by affecting protein structure. The effect may be neutral, beneficial or detrimental, or both beneficial and detrimental, depending on the circumstances. For example, a heterozygous sickle cell mutation confers resistance to malaria, but a homozygous sickle cell mutation is usually lethal. Other polymorphisms occur in noncoding regions but may exert phenotypic effects indirectly via influence on replication, transcription, and translation. A single polymorphism may affect more than one phenotypic trait. Likewise, a single phenotypic trait may be affected by polymorphisms in different genes. Further, some polymorphisms predispose an individual to a distinct mutation that is causally related to a certain phenotype.

Phenotypic traits include diseases that have known but hitherto unmapped genetic components (e.g., agammaglobulimenia, diabetes insipidus, Lesch-Nyhan syndrome, muscular dystrophy, Wiskott-Aldrich syndrome, Fabry's disease, familial hypercholesterolemia, polycystic kidney disease, hereditary spherocytosis, von Willebrand's disease, tuberous sclerosis, hereditary hemorrhagic telangiectasia, familial colonic polyposis, Ehlers-Danlos syndrome, osteogenesis imperfecta, and acute intermittent porphyria). Phenotypic traits also include symptoms of, or susceptibility to, multifactorial diseases of which a component is, or may be, genetic, such as autoimmune diseases, inflammation, cancer, diseases of the nervous system, and infection by pathogenic microorganisms. Some examples of autoimmune diseases include rheumatoid arthritis, multiple sclerosis, diabetes (insulin-dependent and non-independent), systemic lupus erythematosus and Graves disease. Some examples of cancers include cancers of the bladder, brain, breast, colon, esophagus, kidney, leukemia, liver, lung, oral cavity, ovary, pancreas, prostate, skin, stomach and uterus. Phenotypic traits also include characteristics such as longevity, appearance (e.g., baldness, obesity), strength, speed, endurance, fertility, and susceptibility or receptivity to particular drugs or therapeutic treatments.

Correlation is performed for a population of individuals who have been tested for the presence or absence of one or more phenotypic traits of interest and for polymorphic profile. The alleles of each polymorphism in the profile are then reviewed to determine whether the presence or absence of a particular allele is associated with the trait of interest. Correlation can be performed by standard statistical methods such as a κ-squared test and statistically significant correlations between polymorphic form(s) and phenotypic characteristics are noted. For example, it might be found that the presence of allele A1 at polymorphism A correlates with heart disease. As a further example, it might be found that the combined presence of allele A1 at polymorphism A and allele B1 at polymorphism B correlates with increased risk of cancer.

Such correlations can be exploited in several ways. In the case of a strong correlation between a set of one or more polymorphic forms and a disease for which treatment is available, detection of the polymorphic form set in a human or animal patient may justify immediate administration of treatment, or at least the institution of regular monitoring of the patient. Detection of a polymorphic form(s) correlated with serious disease in a couple contemplating a family may also be valuable to the couple in their reproductive decisions. For example, the female partner might elect to undergo in vitro fertilization to avoid the possibility of transmitting such a polymorphism from her husband to her offspring. In the case of a weaker, but still statistically significant correlation between a polymorphic set and human disease, immediate therapeutic intervention or monitoring may not be justified. Nevertheless, the patient can be motivated to begin simple life-style changes (e.g., diet, exercise) that can be accomplished at little cost to the patient but confer potential benefits in reducing the risk of conditions to which the patient may have increased susceptibility by virtue of variant alleles. Identification of a polymorphic profiles in a patient correlated with enhanced receptiveness to one of several treatment regimes for a disease indicates that this treatment regime should be followed.

For animals and plants, correlations between polymorphic profiles and phenotype are useful for breeding for desired characteristics. For example, Beitz et al., U.S. Pat. No. 5,292,639 discuss use of bovine mitochondrial polymorphisms in a breeding program to improve milk production in cows. To evaluate the effect of mtDNA D-loop sequence polymorphism on milk production, each cow was assigned a value of 1 if variant or 0 if wildtype with respect to a prototypical mitochondrial DNA sequence at each of 17 locations considered. Each production trait was analyzed individually with the following animal model:

Yijkpn=μ+YSi+Pj+Xk+β1+ . . . β17+PEn+an+ep where Yijknp is the milk, fat, fat percentage, SNF, SNF percentage, energy concentration, or lactation energy record; μ is an overall mean; YSi is the effect common to all cows calving in year-season; Xk is the effect common to cows in either the high or average selection line; β1 to β17 are the binomial regressions of production record on mtDNA D-loop sequence polymorphisms; PEn is permanent environmental effect common to all records of cow n; an is effect of animal n and is composed of the additive genetic contribution of sire and dam breeding values and a Mendelian sampling effect; and ep is a random residual. It was found that eleven of seventeen polymorphisms tested influenced at least one production trait. Bovines having the best polymorphic forms for milk production at these eleven loci are used as parents for breeding the next generation of the herd.

b. Forensics

Determination of which polymorphic forms occupy a set of polymorphic sites in an individual identifies a set of polymorphic forms that distinguishes the individual. See generally National Research Council, The Evaluation of Forensic DNA Evidence (Eds. Pollard et al., National Academy Press, DC, 1996). The more sites that are analyzed the lower the probability that the set of polymorphic forms in one individual is the same as that in an unrelated individual.

The capacity to identify a distinguishing or unique set of forensic markers in an individual is useful for forensic analysis. For example, one can determine whether a blood sample from a suspect matches a blood or other tissue sample from a crime scene by determining whether the set of polymorphic forms occupying selected polymorphic sites is the same in the suspect and the sample. If the set of polymorphic markers does not match between a suspect and a sample, it can be concluded (barring experimental error) that the suspect was not the source of the sample. If the set of markers does match, one can conclude that the DNA from the suspect is consistent with that found at the crime scene. If frequencies of the polymorphic forms at the loci tested have been determined (e.g., by analysis of a suitable population of individuals), one can perform a statistical analysis to determine the probability that a match of suspect and crime scene sample would occur by chance.

p(ID) is the probability that two random individuals have the same polymorphic or allelic form at a given polymorphic site. In diallelic loci, four genotypes are possible: AA, AB, BA, and BB. If alleles A and B occur in a haploid genome of the organism with frequencies x and y, the probability of each genotype in a diploid organism are (see WO 95/12607):

Homozygote: p(AA)=x2

Homozygote: p(BB)=y2=(1−x)2

Single Heterozygote: p(AB)=p(BA)=xy=x(1−x)

Both Heterozygotes: p(AB+BA)=2xy=2x(1−x)

The probability of identity at one locus (i.e., the probability that two individuals, picked at random from a population will have identical polymorphic forms at a given locus) is given by the equation:

p(ID)=(x2)2 +(2xy)2+(y2)2.

These calculations can be extended for any number of polymorphic forms at a given locus. For example, the probability of identity p(ID) for a 3-allele system where the alleles have the frequencies in the population of x, y and z, respectively, is equal to the sum of the squares of the genotype frequencies:

p(ID)=x4+(2xy)2+(2yz)2+(2xz)2+z4+y4

In a locus of n alleles, the appropriate binomial expansion is used to calculate p(ID) and p(exc).

The cumulative probability of identity (cum p(ID)) for each of multiple unlinked loci is determined by multiplying the probabilities provided by each locus.

cum p(ID)=p(ID1)p(ID2)p(ID3) . . . p(IDn)

The cumulative probability of non-identity for n loci (i.e. the probability that two random individuals will be different at 1 or more loci) is given by the equation:

cum p(nonID)=1−cum p(ID).

If several polymorphic loci are tested, the cumulative probability of non-identity for random individuals becomes very high (e.g., one billion to one). Such probabilities can be taken into account together with other evidence in determining the guilt or innocence of the suspect.

B. Paternity Testing

The object of paternity testing is usually to determine whether a male is the father of a child. In most cases, the mother of the child is known and thus, the mother's contribution to the child's genotype can be traced. Paternity testing investigates whether the part of the child's genotype not attributable to the mother is consistent with that of the putative father. Paternity testing can be performed by analyzing sets of polymorphisms in the putative father and the child.

If the set of polymorphisms in the child attributable to the father does not match the putative father, it can be concluded, barring experimental error, that the putative father is not the real father. If the set of polymorphisms in the child attributable to the father does match the set of polymorphisms of the putative father, a statistical calculation can be performed to determine the probability of coincidental match.

The probability of parentage exclusion (representing the probability that a random male will have a polymorphic form at a given polymorphic site that makes him incompatible as the father) is given by the equation (see WO 95/12607):

p(exc)=xy(1−xy)

where x and y are the population frequencies of alleles A and B of a diallelic polymorphic site.

(At a triallelic site p(exc) xy(1−xy)+yz(1−yz)+xz(1−xz)+3xyz(1−xyz))), where x, y and z and the respective population frequencies of alleles A, B and C).

The probability of non-exclusion is

p(non-exc)=1−p(exc)

The cumulative probability of non-exclusion (representing the value obtained when n loci are used) is thus:

cum p(non-exc)=p(non-exc1)p(non-exc2)p(non-exc3) . . . p(non-excn)

The cumulative probability of exclusion for n loci (representing the probability that a random male will be excluded)

cum p(exc)=1−cum p(non-exc).

If several polymorphic loci are included in the analysis, the cumulative probability of exclusion of a random male is very high. This probability can be taken into account in assessing the liability of a putative father whose polymorphic marker set matches the child's polymorphic marker set attributable to his/her father.

2. Expression Analysis

The quantitative monitoring of expression levels for large numbers of genes can prove valuable in elucidating gene function, exploring the causes and mechanisms of disease, and for the discovery of potential therapeutic and diagnostic targets. Expression monitoring can be used to monitor the expression (transcription) levels of nucleic acids whose expression is altered in a disease state. For example, a cancer can be characterized by the overexpression of a particular marker such as the HER2 (c-erbB-2/neu) protooncogene in the case of breast cancer.

Expression monitoring can be used to monitor expression of various genes in response to defined stimuli, such as a drug. This is especially useful in drug research if the end point description is a complex one, not simply asking if one particular gene is overexpressed or underexpressed. Therefore, where a disease state or the mode of action of a drug is not well characterized, the expression monitoring can allow rapid determination of the particularly relevant genes.

In arrays of random probes (sometimes known as generic arrays), the hybridization pattern is also a measure of the presence and abundance of relative mRNAs in a sample, although it is not immediately known, which probes correspond to which mRNAs in the sample. However the lack of knowledge regarding the particular genes does not prevent identification of useful therapeutics. For example, if the hybridization pattern on a particular generic array for a healthy cell is known and significantly different from the pattern for a diseased cell, then libraries of compounds can be screened for those that cause the pattern for a diseased cell to become like that for the healthy cell. This provides a detailed measure of the cellular response to a drug.

Generic arrays can also provide a powerful tool for gene discovery and for elucidating mechanisms underlying complex cellular responses to various stimuli. For example, generic arrays can be used for expression fingerprinting. Suppose it is found that the mRNA from a certain cell type displays a distinct overall hybridization pattern that is different under different conditions (e.g., when harboring mutations in particular genes, in a disease state). Then this pattern of expression (an expression fingerprint), if reproducible and clearly differentiable in the different cases can be used as a very detailed diagnostic. It is not required that the pattern be fully interpretable, but just that it is specific for a particular cell state (and preferably of diagnostic and/or prognostic relevance).

Both customized and generic arrays can be used in drug safety studies. For example, if one is making a new antibiotic, then it should not significantly affect the expression profile for mammalian cells. The hybridization pattern can be used as a detailed measure of the effect of a drug on cells, for example, as a toxicological screen.

The sequence information provided by the hybridization pattern of a generic array can be used to identify genes encoding mRNAs hybridized to an array. Such methods can be performed using DNA tags of the invention as the target nucleic acids described in WO 97/27317. DNA tags can be denatured forming first and second tag strands. The denatured first and second tag strands are then hybridized to the complementary regions of the probes, using standard conditions described in WO 97/27317. The hybridization pattern indicates which probes are complementary to tag strands in the sample. Comparison of the hybridization pattern of two samples indicates which probes hybridize to tag strands that derive from mRNAs that are differentially expressed between the two samples. These probes are of particular interest, because they contain complementary sequence to MRNA species subject to differential expression. The sequence of such probes is known and can be compared with sequences in databases to determine the identity of the full-length MRNAs subject to differential expression provided that such mRNAs have previously been sequenced. Alternatively, the sequences of probes can be used to design hybridization probes or primers for cloning the differentially expressed mRNAs. The differentially expressed mRNAs are typically cloned from the sample in which the MRNA of interest was expressed at the highest level. In some methods, database comparisons or cloning is facilitated by provision of additional sequence information beyond that inferable from probe sequence by template dependent extension as described above.

### EXAMPLES

1. Preparation DNA from BAC & PAC Clone

This example provides a method of preparing genomic DNA suitable for use in any of the methods of reducing sample complexity described above that involves use of genomic DNA.

Reagents Used:

1. Qiagen Large-Construct Kit (cat.# 12462)

2. Molecular Biology Grade Water from Bio Whittaker, Cat.#16-01Y

3. Ethanol (Rossville Gold Shield 200 proof)

4. LB Broth Base (Gibco BRL cat #12780-052) 20 grams per liter of DI water will give 1L of LB.

5. Bacto-Agar (Difco cat #0140-01) 15 grams per 1L of LB.

6. Antibiotics: Chloramphenicol and Kanamycine (25 ug/ml)

Protocol:

1. Streak some of the “stab” clone to a plate of LB agar containing 25 μg/ml chloramphenicol or kanamycin. Grow at 37° C. for overnight.

2. Pick colony with a pipet tip and shoot the entire tip into 5 ml LB with antibiotics (25 ug/ml kanamycin for PAC and 25 ug/ml chloramphenicol for BAC). Put in a shaking incubator at 300 rpm overnight @37Co

3. Centrifuge (SM24 Rotor) at 3,000 rpm for 10 minute in the Sorvall.

4. Discard supernatants. Add 1.250 ml of Qiagen buffer “P1” to sample. Resuspend each pellet by pipetting up and down several times until no cell clumps remain.

5. Add 1.250 ml of Qiagen buffer “P2” to sample and gently shake tube to mix the contents. Let sit at room temperature for 5 min. The appearance of the suspension should change from very turbid to almost translucent.

6. Add 1.250 ml of Qiagen buffer “P3” to each tube and gently shake during addition. A thick white precipitate of protein and E. coli DNA will form. Place the tubes on ice for at least 5 min.

7. Spin sample at 10,000 rpm for 10 minutes at 4° C. in the SM24 rotor.

8. Remove tubes from centrifuge and place on ice. Transfer the supernatant using a disposable pipette into a new tube and precipitate the DNA with 2 volumes of ethanol. Mix by inverting tube a few times; place tubes on ice for at least 5 minutes.

9. Spin sample at 10,000 rpm for 10 min at 4° C. in the SM24 rotor.

10. Carefully pour off the ethanol. Wash the pellet with 75% ethanol and centrifuge at 10,000 rpm for 5 minute twice.

11. Place the tubes upside down on a paper towel to let the ethanol wick off the sides of the tube. Let air dry for 5-10 min until the DNA pellets turn from white to translucent in appearance that means most of the ethanol has evaporated.

12. Add 250 μl TE or water into DNA sample, allow the solution to sit in the tube on ice for about 30 minutes.

13. Determine DNA concentration by using both UV spectrophotometry and quantitative analysis on an agarose gel.

2. Fragmentation and Labeling:

This example illustrates how to fragment genomic DNA by DNaseI digestion, and how to label the resulting fragments with biotin.

1. Put 30 ug BAC or PAC DNA:

2 ul of diluted DNaseI (0.1 U/ul, 10 times dilution)

7.4 10× One-Phor-All Buffer PLUS

Make up volume to 74 ul

2. Incubate at:

37° C. for 10 min

99° C. for 10 min

Place on ice for 2 min

3. Spin briefly.

6. Add:

2 μl of Boehringer Mannheim or BRL TdT (25 U/μl)

1 μl of biotin ddATP (1 mM)

7. Pipettomix.

8. Incubate at:

37° C. for 90 min

99° C. for 10 min

25° C. indefinitely.

3. Fragmentation and Labeling of PCR Fragments

This example illustrates an alternative procedure for fragmenting by DNaseI digestion and labelling that is particularly suitable for use with long range PCR products.

1. Start with purified Long Range PCR products in final volume of 300-350 uL. The concentration of DNA is determined by OD 260 measurement.

2. 280 μg DNA can be labelled to give a final target concentration of 5-10 pM for a complexity range of 3-6 MB.

3. The labeling is performed in 5 independent Eppenderf tubes with each one containing:

| | |
|---|---|
| 10X One-Phor-All Buffer PLUS | 37 μL |
| Gibco DNaseI (at 0.5 U/uL; | 2 uL |
| 1 uL Dnase 1 + 1 uL | |
| 10 mM Tris, pH 8) | |
| Purified LR-PCR products up to | 331 uL |
| Total reaction volume (5Mb DNA | |
| need 280 ug): | 370 uL |

| 4. | Tap to mix. | |
|---|---|---|
| 5. | Incubate at: | 37° C. for 10 min |
| | | 99° C. for 10 min |
| | | 25° C. for <5 min |

| | | |
|---|---|---|
| 6. | Spin briefly. | |
| 7. | Add: | |
| | Boehringer Mannheim TdT (25 U/μl) | 20 μL |
| | Biotin ddATP (1 mM) | 20 μL |
| 8. | Tap to mix. | |

| | | |
|---|---|---|
| 9. | Incubate at: | 37° C. for 90 min |
| | | 99° C. for 10 min |
| | | 25° C. for <5 min |

5. Removal of Repeat Sequences

This example illustrates DNase digestion of human placenta DNA, denaturation and annealing of fragments.

Human Placenta DNA is Digested with DNaseI as Follows:

160 μg Human Placenta DNA (0.08 fm for the full length)

in 220 μL for fragmentation [64 μL DNA (2.5 ug/μL), 22 μL 10× buffer, 3.5 μL DNaseI (0.35 U), 132 μL wafer]

9 μL 480 mM NaPO₄ Buffer, pH 7.4 is added to reach final NaPO₄ conc. at 126 mM and a volume of 301L. The sample is denatured for 5 min at 99° C. The mixture is incubated at 65° C., for 90 min to allow repeat sequence to hybridize. The mixture is then diluted to 10 mM NaPO₄ for HPLC.

6. HPLC Hvdroxyapatite Chromatography

This example illustrates use of a hydroxyapatite column to separate single stranded and double stranded DNA. This experiment used single stranded fragments, average length 60 bases, from chromosome 21 and double stranded fragments of herring sperm DNA (average length 500 bp). Both single and double stranded DNA were present at 9 uM. The column was an Econo-Pac CHT-II Cartridge having a DNA capacity of 160 ug. The column was loaded with DNA in 10 mM phosphate. At 10-20 mM phosphate hydroxyapatite binds both single and double stranded DNA. DNA was then eluted at a gradient from 10 mM to 1 M NaPO₄ Buffer, pH 7.4 over 30 min. Elution was monitored by absorbance at 260 nm. At 5 min, there was a small peak indicating release of single stranded DNA, and at 25 min there was a larger peak indicating release of double stranded DNA, as shown in FIG. 1.

7. Preparing Target DNA for Hybridization

1. After reducing sample complexity and optionally labelling, target DNA can be prepared for application to a chip as follows:

Final Concentration into chip

| | | |
|---|---|---|
| TMACL: 3M | 177 ul | 5M |
| Tris (pH 7.8 or 8): 10 mM | 3 ul | 1M |
| Triton X-100: 0.01% | 3 ul | 1% |
| Herring Sperm DNA: 100 ug/ml | 3 ul | 10 mg/ml |
| Control Oligo b948: 50 pM | 3 ul | 5 nM (optional) |

Labeled DNA & H₂O (˜110 ul) or up to 300 ul final volume

The concentration of labelled DNA can range from about 0.1 pM to 100 pM.

2. Denature the sample at 99° C. for 5 min and spin down.

3. Warm up the chip at 40° C. about 20 minutes before adding the hybridization mixture.

4. Add sample into chip (vented with P-10 pipet tip).

Put chip into an oven at 40° C. and rotisserie overnight at speed of 40 rpm to allow hybridization to occur.

8. Staining and Scanning a Chip

This example illustrates a procedure for detecting hybridization of sample to probes on a chip.

A. Solutions:

1. Streptavidin-Phycoerythrin Solution

1 ml total (300 ul/chip) 470 ul water

500 ul 2× MES

20 μl acetylated BSA(50 mg/ml)

10 ul streptavidin-phycoerythrin(1 mg/ml)

2. Antibody Solution

1 ml total (300 ul/chip)

470 ul water

500 ul 2× MES

20 ul acetylated BSA(50 mg/ml)

10 ul biotinylated anti-streptavidin(1 mg/ml)

B. Procedures

1. Prime a fluidics station (available from Affymetrix, Inc., Santa Clara) with 6× SSPE/0.01% Triton X-100. Also turn on a scanner (also available from Affymetrix) and create an experimental information file according to the manufacturer's instructions.

2. Remove and save hybridization solution from chip and store at −20° C.

3. Rinse chip twice with 1× MES, 0.01% Triton X-100

4. Add 300 μl streptavidin solution to chip and twirl at a speed of 40 rpm on the rotisserie at room temperature for 20 min.

5. Remove stain solution and rinse 2× with 1× MES, 0.01% Triton X-100.

6. Add 300 μl antibody solution to chip and twirl at a speed of 40 rpm on the rotisserie at room temperature for 20 min.

7. Remove antibody solution and rinse 2× with 1× MES, 0.01% Triton X-100.

8. Add 300 μl staining solution again to chip and twirl at a speed of 40 rpm on the rotisserie at room temperature for 20 min.

9. Insert chip into fluidics station and wash 6 times at 35° C. with 6× SSPE, 0.01% Triton X-100.

10. Breathe some moisture onto the chip surface and wipe clean. Insert chip into scanner.

11. Set up the scanner at settings “slowav6k” and “scan6k” at 2.27 μm pixel size with 560 nm wavelength.

12. Scan the chip.

Additional methodology useful for practicing the invention are described in Birren et al. supra. All publications and patent applications cited above are incorporated by reference in their entirety for all purposes to the same extent as if each individual publication or patent application were specifically and individually indicated to be so incorporated by reference. Although the present invention has been described in some detail by way of illustration and example for purposes of clarity and understanding, it will be apparent that certain changes and modifications may be practiced within the scope of the appended claims.

What is claimed is:

1. A method of analyzing a subset of nucleic acids within a nucleic acid population, comprising:
(a) providing a population of nucleic acid fragments at least some of which have sequences that are repeated more than once in a genome;
(b) incubating single stranded forms of the population of nucleic acid fragments under annealing conditions, whereby single stranded forms of nucleic acid fragments having repeat sequences preferentially hybridize to each other relative to nucleic acid fragments lacking repeat sequences;
(c) separating single stranded forms of the population of nucleic acid fragments from annealed double stranded forms, the single stranded forms being enriched for nucleic acid fragments lacking repeat sequence;
(d) hybridizing the separated single stranded forms of the population of nucleic acid fragments to a nucleic acid probe array,
(e) determining hybridization of the probes to the single stranded forms of the population of nucleic acid fragments, thereby analyzing the fragments.

2. The method of claim 1, wherein the population of nucleic acid fragments are genomic fragments.

3. The method of claim 1, wherein the population of nucleic acid fragments are from the human genome.

4. The method of claim 1, wherein the population of nucleic acids are from the same chromosome of the human genome.

5. The method of claim 4, wherein the population of nucleic acid fragments span the chromosome of the human genome.

6. The method of claim 1, further comprising denaturing the population of nucleic acids fragments before the incubating step.

7. The method of claim 1, wherein the separating step is performed by column chromatography.

8. The method of claim 7, wherein the column is a hydroxyapatite column.

9. The method of claim 8, wherein the separating step is performed under conditions whereby annealed and single stranded fragments elute in different fractions from the column.

10. The method of claim 1, wherein the separating step is performed by HPLC.

11. The method of claim 1, wherein the separating step is performed by successively performing hydroxyapatite chromatography and HPLC.

12. The method of claim 1, wherein the probe array comprises a set of probes complementary to a known reference sequence, the reference sequence being the same or a variant of the sequence of a nucleic acid from which the population of nucleic acid fragments was obtained.

13. The method of claim 12, wherein the determining indicates the presence of at least one variation in a fragment hybridized to the array relative to the reference sequence.

14. The method of claim 12, wherein the population of nucleic acids are from a chromosome from a first individual, and the reference sequences is that of a corresponding chromosome from a second individual.

15. A method of analyzing a subset of nucleic acids within a nucleic acid population, comprising:
(a) providing driver and tester populations of nucleic acids;
(b) hybridizing the driver and tester populations with each other;
(c) separating nucleic acids from the tester population that hybridize to the driver population from tester nucleic acids that do not hybridize;
(d) hybridizing either the tester nucleic acids that do hybridize to the driver population, or the tester nucleic acids that do not hybridize to the driver population to a nucleic acid probe array;
(e) determining hybridization of the probes to the tester nucleic acids thereby analyzing the tester nucleic acids.

16. The method of claim 15, wherein the driver population of nucleic acids each bear a tag by which the driver population of nucleic acids can be immobilized to a binding moiety with affinity for the tag.

17. The method of claim 16, wherein the tag is biotin, and the binding moiety is avidin or streptavidin.

18. The method of claim 17, wherein the separating step is performed by immobilizing the driver population of nucleic acids and tester population of nucleic acids hybridized to the driver population via the tags of the driver population.

19. The method of claim 15, wherein the driver population of nucleic acids are a population of genomic DNA fragments, and the tester nucleic acids are a population of mRNA or nucleic acids derived therefrom, and the method further comprises denaturing tester nucleic acids from the driver population of nucleic acids after step (b), the resulting tester nucleic acids showing reduced variance in copy number between different fragments than in the population of MRNA or nucleic acids derived therefrom; and wherein the resulting tester nucleic acids are hybridized to the array.

20. The method of claim 15, wherein the driver population of nucleic acids are genomic DNA from a first source, and the tester population of nucleic acids are genomic DNA from a second source, and the method further comprises denaturing nucleic acids of the tester population from the driver population of nucleic acids after step (b), the resulting tester nucleic acids being enriched for tester nucleic acids having common sequences with the driver population of nucleic acids relative to the population of tester nucleic acids, and wherein the resulting tester nucleic acids are hybridized to the array.

21. The method of claim 20, wherein the tester population of nucleic acids are from a genome, and the driver population of nucleic acids are from at least one region of the genome, or a variant thereof from the same species as the genome.

22. The method of claim 21, wherein the at least one region is a PCR amplification product.

23. The method of claim 21, wherein the at least one region is cloned into a BAC, YAC or PAC.

24. The method of claim 21, wherein the driver population of nucleic acids are from a plurality of noncontiguous regions of the genome or the variant thereof.

25. The method of claim 21, wherein the driver population of nucleic acids are from at least ten noncontiguous regions of the genome or the variant thereof.

26. The method of claim 21, wherein the method is repeated for a further population of tester nucleic acids from a further source.

27. The method of claim 21, wherein the method is repeated for at least ten further populations of tester nucleic acids from at least ten further sources.

28. The method of claim 27, wherein the at least ten further sources are from ten individuals in the same species.

29. The method of claim 28, wherein the species is human.

30. The method of claim 15, wherein the driver population of nucleic acids are genomic DNA from a first source, and the tester population of nucleic acids are genomic DNA from a second source, and the tester nucleic acids that do not hybridize to the driver fragments are hybridized to the array, these tester nucleic acids being enriched for nucleic acids having sequences not common with sequences of the nucleic acids in the driver population.

31. The method of claim 15, wherein the driver population of nucleic acids are mRNA or nucleic acids derived therefrom, and the tester population of nucleic acids are genomic DNA; and the method further comprises denaturing tester nucleic acids from the driver population after step (b), the resulting tester nucleic acids being enriched for genomic sequences that hybridize to the MRNA; and wherein the resulting tester nucleic acids are hybridized to the nucleic acid probe array.

32. The method of claim 15, wherein the population of driver nucleic acids are mRNA or nucleic acids derived therefrom from a first source, and the population of tester nucleic acids are mRNA or nucleic acids derived therefrom from a second source, and the method further comprises denaturing tester nucleic acids from the driver nucleic acids after step (b), the resulting tester nucleic acids being enriched for nucleic acids common to the two sources, and wherein the resulting tester nucleic acids are hybridized to the nucleic acid probe array.

33. The method of claim 32, wherein the first and second source are from the same tissue of different species.

34. The method of claim 32, wherein the first and second source are from different tissues of the same species.

35. The method of claim 15, wherein the population of driver nucleic acids are mRNA or nucleic acids derived therefrom from a first source, and the population of tester nucleic acids are mRNA or nucleic acids derived therefrom from a second sources, the tester nucleic acids that do not hybridize with the driver nucleic acids are hybridized to the array, these tester nucleic acids s being enriched for sequence present in the second source and absent in the first source.

36. The method of claim 35, wherein the first and second source are from the same tissue of different species.

37. The method of claim 35, wherein the first and second source are from different tissues of the same species.
